(19) United States
(12) Patent Application Publication
(10) Pub. No.: US 2020/0185073 A1
(43) Pub. Date: Jun. 11, 2020

(54) SYSTEM AND METHOD FOR PROVIDING PERSONALIZED HEALTH DATA

(71) Applicant: DOC.AI Incorporated, Palo Alto, CA (US)

(72) Inventor: Walter DE BROUWER, Los Altos, CA (US)

(21) Appl. No.: 16/705,076

(22) Filed: Dec. 5, 2019

Related U.S. Application Data
(60) Provisional application No. 62/775,782, filed on Dec. 5, 2018.

(51) Int. Cl. G16H 15/00; G06N 3/02; G16H 10/60

(52) U.S. Cl. CPC G16H 15/00; G16H 10/60; G06N 3/02

(57) ABSTRACT

A method for providing personalized blood tests is provided, the method comprising receiving health data associated with a user and an input associated with the user. Based on the received user input, parameters associated with the user may be predicted via a trained neural network model. The received health data and the predicted parameters may be stored, using block chain, in decentralized nodes. The received health data and the predicted parameters may be transmitted to a remote device to personalize the health data. The method may further comprise receiving personalized health data associated with the user and at least one predictive model based on the personalized health data. The personalized health data may comprise the received health data filtered by the predicted parameters, and the predictive model may be configured to predict future health-related information. The personalized health data and the future health-related information may be displayed on a graphical user interface.

### CROSS REFERENCE TO RELATED APPLICATIONS

This application is based upon and claims priority to U.S. Provisional Application No. 62/775,782 filed Dec. 5, 2018, the entire contents of which are incorporated herein by reference.

### TECHNICAL FIELD

Embodiments of the present disclosure relate to systems and methods for providing personalized health data. In particular, the embodiments of the present disclosure relate to using machine learning algorithms to provide precise and personalized blood tests.

### BACKGROUND

In 2018, the global market for blood testing has reached $90.21 billion. 40% of the total revenue for blood testing comes from North America. In the United States, diagnostic tests are performed 9 billion times a year. In such a world where medicine has become a continuous function, conventional systems and methods for providing healthcare data, such as blood test results, are not personalized.

Conventional blood panels provide information, including levels of complete blood count (CBC), lipidome (total cholesterol, high-density lipoprotein (HDL), low-density lipoprotein (LDL), triglycerides, lipoprotein-a, apolipoprotein B), thyroid-stimulating hormone (TSH), glucose, glycated hemoglobin (HA1c), creatinine, testosterone, cortisol, high-sensitivity C-reactive protein (hs-CRP), ferritin, and folate. While Direct Access Testing (DAT) allows consumers to initiate blood testing and choose the tests they would like from a limited menu, the results of DAT are still not personalized to the user. For example, an average value or value range that is deemed “normal” in certain panel elements for a certain group of people (group 1, namely) with certain genetic trait may be higher than a second average value or value range that is deemed “normal” in those panel elements for another group of people (group 2, namely) without the certain genetic trait. In this situation, the slightly “higher” value of people in group 1 would be reflected as “abnormal,” even though it may be completely normal for the people in group 1 with the certain genetic trait. Accordingly, these types of situations would lead to false positives of various diseases.

One possible solution to overcome the false positive situations is to take multiple repeating blood tests throughout the year. That is, testing a parameter repeatedly will reduce the chances of false positives. However, repeating blood tests can be costly and can be a waste of medical resources, which are extremely limited and not readily available to a majority of the global population.

In view of the above deficiencies, there exists a need to improve the accuracy and precision of the blood test results and personalize the results to the user. In particular, there is a need to filter the results based on certain parameters, such as gender, age, and ethnicity, so that the results can be parsed to provide personalized blood tests. Optimal ranges need to be empirical and dynamic, such that the optimal ranges are adjusted with different input parameters, including gender, age, ethnicity (genome), exposome (places the user has lived), diet, and physiome (activities, lifestyle, etc.). Such improved systems and methods have the potential to increase precision and accuracy of healthcare data provided to the user, thereby reducing the likelihood of false positives and obviating the need to take multiple blood tests a year.

### SUMMARY

In accordance with an exemplary embodiment of the present disclosure, systems and computer-implemented methods are provided for providing a personalized blood test. By way of example, the method comprises receiving, from a digital device, health data associated with a user. The health data may comprise blood test results. The method further comprises receiving, from a digital device, an input associated with the user, predicting, via a trained neural network model, parameters associated with the user based on the received user input. The received health data and the predicted parameters are stored, using block chain, in a plurality of decentralized nodes and transmitted to a remote device. The method further comprises receiving, from the remote device, personalized health data associated with the user, wherein the personalized health data comprises the received health data filtered by the predicted parameters. The method further comprises receiving, from the remote device, at least one predictive model based on the personalized health data, wherein the predictive model is configured to predict future health-related information. The personalized health data and the future health-related information are displayed on a graphical user interface of the digital device.

In some embodiments, the predicted parameters associated with the user may comprise at least one of gender, age, ethnicity, weight, height, or body mass index. Additionally or alternatively, the received user input may comprise at least one of an image of the user, clinome, phenome, exposome, genome, proteome, microbiome, pharmacome, or physiome. The personalized health data may comprise personalized test results that are filtered by gender, age, and ethnicity associated with the user. In other embodiments, the future health-related information may comprise at least one of a number of future healthcare visits the user will have, risks for mortality causes, microbial diversity, healthiest location to live, a number of steps the user will take per day, future potential for weight gain, risk of allergies, or future sleep patterns.

In some embodiments, the method may further comprise displaying, on a graphical user interface, optimal range associated with the personalized health data. The optimal range may be calculated, via a machine-learning algorithm, based on the predicted parameters associated with the user. In yet another embodiment, the method may further comprise aggregating the received health data associated with the user with shared health data received from other users, and updating the predictive model based on the aggregation.

In some aspects, the method may further comprise generating a reward to the user in response to determining that the received health data and the predicted parameters are transmitted to the remote device. The reward may be displayed on the graphical user interface of the digital device. The digital device may comprise at least one of a computer, a laptop, a smartphone, a tablet, or a smartwatch. In yet another embodiment, the method may further comprise encrypting the received health data and the predicted parameters before storing the received health data and the predicted parameters in the plurality of decentralized nodes.

In accordance with another exemplary embodiment of the present disclosure, a non-transitory computer-readable medium comprising instructions is provided. The instructions, when executed by at least one processor, may cause the at least one processor to perform operations. By way of example, the operations may comprise receiving, from a digital device, health data associated with a user. The health data may comprise blood test results. The method further comprises receiving, from a digital device, an input associated with the user, predicting, via a trained neural network model, parameters associated with the user based on the received user input. The received health data and the predicted parameters are stored, using block chain, in a plurality of decentralized nodes and transmitted to a remote device. The method further comprises receiving, from the remote device, personalized health data associated with the user, wherein the personalized health data comprises the received health data filtered by the predicted parameters. The method further comprises receiving, from the remote device, at least one predictive model based on the personalized health data, wherein the predictive model is configured to predict future health-related information. The personalized health data and the future health-related information are displayed on a graphical user interface of the digital device.

In some embodiments, the predicted parameters associated with the user may comprise at least one of gender, age, ethnicity, weight, height, or body mass index. Additionally or alternatively, the received user input may comprise at least one of an image of the user, clinome, phenome, exposome, genome, proteome, microbiome, pharmacome, or physiome. The personalized health data may comprise personalized test results that are filtered by gender, age, and ethnicity associated with the user. In other embodiments, the future health-related information may comprise at least one of a number of future healthcare visits the user will have, risks for mortality causes, microbial diversity, healthiest location to live, a number of steps the user will take per day, future potential for weight gain, risk of allergies, or future sleep patterns.

In some embodiments, the method may further comprise displaying, on a graphical user interface, optimal range associated with the personalized health data. The optimal range may be calculated, via a machine-learning algorithm, based on the predicted parameters associated with the user. In yet another embodiment, the method may further comprise aggregating the received health data associated with the user with shared health data received from other users, and updating the predictive model based on the aggregation.

In some aspects, the method may further comprise generating a reward to the user in response to determining that the received health data and the predicted parameters are transmitted to the remote device. The reward may be displayed on the graphical user interface of the digital device. The digital device may comprise at least one of a computer, a laptop, a smartphone, a tablet, or a smartwatch. In yet another embodiment, the method may further comprise encrypting the received health data and the predicted parameters before storing the received health data and the predicted parameters in the plurality of decentralized nodes.

Additional objects and advantages of the embodiments of the present disclosure will be set forth in part in the description which follows, and in part will be obvious from the description, or may be learned by practice of the embodiments of the present disclosure.

It is to be understood that both the foregoing general description and the following detailed description are exemplary and explanatory only and are not restrictive of the disclosed embodiments, as claimed.

### BRIEF DESCRIPTION OF THE DRAWINGS

The accompanying drawings, which are incorporated in and constitute a part of this present disclosure, illustrate disclosed embodiments of the present disclosure and, together with the description, serve to explain the principles of the present disclosure. In the drawings:

FIG. 1 shows an exemplary schematic diagram of a system for providing a personalized blood test, in accordance with the embodiments of the present disclosure;

FIG. 2 shows an exemplary schematic diagram of a device for providing a personalized blood test, in accordance with the embodiments of the present disclosure;

FIG. 3A shows an exemplary graphical user interface displayed on an exemplary digital device, in accordance with the embodiments of the present disclosure;

FIG. 3B shows another exemplary graphical user interface displayed on an exemplary digital device, in accordance with the embodiments of the present disclosure;

FIG. 4 shows a flowchart of an exemplary process of providing a personalized blood test, in accordance with the embodiments of the present disclosure;

FIG. 5 shows an exemplary schematic diagram of a regression deep learning convolutional neural network model of age, in accordance with the embodiments of the present disclosure; and

FIG. 6 shows an exemplary schematic diagram of a regression deep learning convolutional neural network model to predict height and weight, in accordance with the embodiments of the present disclosure.

### DETAILED DESCRIPTION

Reference will now be made in detail to the disclosed embodiments, examples of which are illustrated in the accompanying drawings. Wherever convenient, the same reference numbers will be used throughout the drawings to refer to the same or like parts. The disclosed embodiments include methods and systems configured to provide, for example, a personalized blood test. It should be appreciated, however, that the present disclosure is not limited to these specific embodiments and details, which are exemplary only. For example, the methods and systems in the disclosed embodiments may be configured to provide other personalized health data and is not limited to providing personalized blood tests. It is further understood that one possessing ordinary skill in the art, in light of known systems and methods, would appreciate the use of the embodiments of the present disclosure for its intended purposes and benefits in any number of alternative embodiments, depending on specific design and other needs.

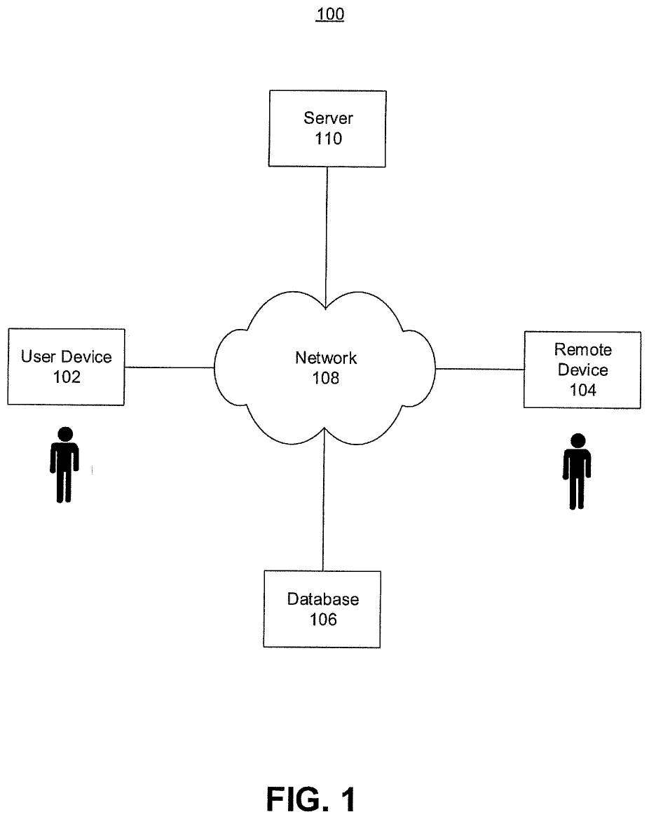

FIG. 1 shows an exemplary schematic diagram of a system 100 for providing a personalized blood test, in accordance with the embodiments of the present disclosure. The arrangement and number of components in system 100 is provided for purposes of illustration. Additional arrangements, number of components, and other modifications may be made, consistent with the present disclosure.

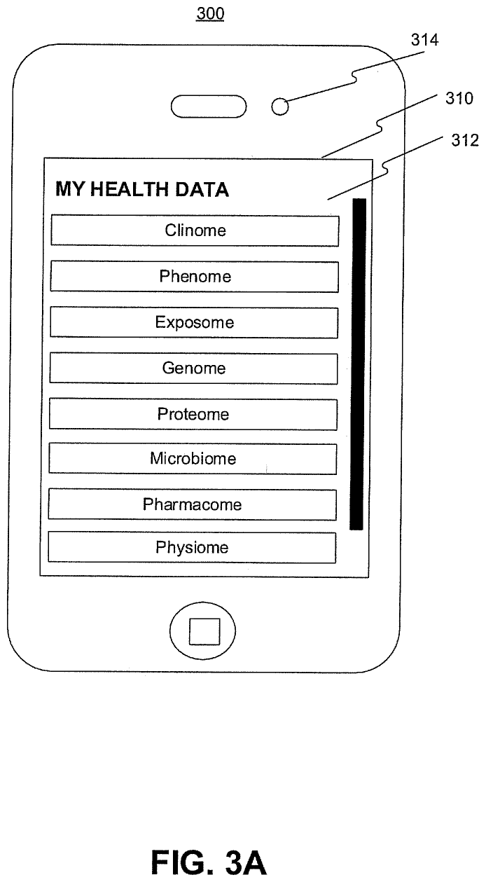

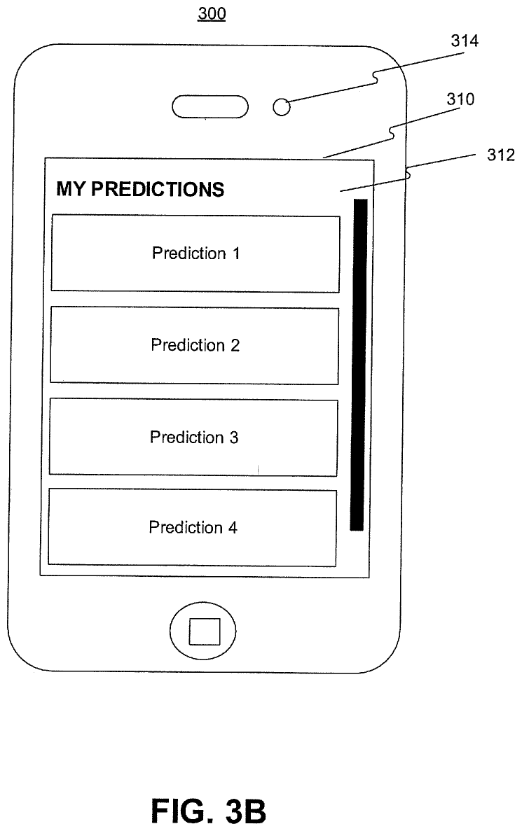

As shown in FIG. 1, in some embodiments, system 100 may include a user device 102, a remote device 104, a database 106, a network 108, and a server 110. The device 102 may be associated with a user. The user may operate a user device 102 to communicate to and/or through network 106 with other components of system 100, such as a remote device 104 and/or the database 106, via network 108 and/or server 110. By way of example, the user device 102 may include one or more devices such as a smartphone, a tablet, a netbook, an electronic reader, a pair of electronic glasses, a smart band, a smart watch, a personal digital assistant, a personal computer, a laptop computer, a pair of multifunctional glasses, a tracking device, a wearable device, a virtual reality headset, or other types of electronics or communication devices. In some exemplary embodiments, the user device 102 may be configured to execute an application (for example, application 312 in FIGS. 3A-3B), which may be configured to allow the user to input health data associated with a user. In some embodiments, the user device 102 may include one or more sensors (not shown), such as any type of image sensor. For example, the user device 102 may include a camera.

A remote device 104 may be associated with healthcare technicians, research technicians, hospitals, doctors, data scientists, service providers, or any other type of entity that gathers healthcare data, develops machine learning algorithms, develops predictive models, analyzes data, etc. The remote device 104 may be operated to communicate with other components of system 100, such as a first device 102 and/or database 106, via network 108 and/or server 110. By way of example, the remote device 104 may include electronic devices such as a smartphone, a tablet, a netbook, an electronic reader, a pair of electronic glasses, a smart band, a smart watch, a personal digital assistant, a personal computer, a laptop computer, a pair of multifunctional glasses, a tracking device, a wearable device, a virtual reality headset, or other types of electronics or communication devices. In some exemplary embodiments, the remote device 104 may be configured to execute an application (for example, application 312 in FIGS. 3A-3B), which may be configured to allow one or more users operating the remote device 104 to request and/or receive health data associated with the user associated with the user device 102. The remote device 104 may also be able to request and/or receive an image of the user associated with the user device 102. The remote device 104 may be configured to communicate with the database 106 through the network 108 to receive and/or store information associated with the user. The remote device 104 may be configured to receive and/or store information associated with the user automatically or upon request. In some embodiments, the remote device 104 may include one or more sensors (not shown), such as any type of image sensor. For example, the remote device 104 may include a camera.

System 100 may also include a database 106, which may include one or more memory devices that store information and are accessed through network 108. By way of example, database 106 may include Oracle™ databases, Sybase™ databases, or other relational databases or non-relational databases, such as Hadoop sequence files, HBase, or Cassandra. Database 106 may include, for example, user's healthcare data, parameters associated with the user, predictive models, etc. Additionally or alternatively, the data stored in the database 106 may take or represent various forms including, but not limited to, images videos, documents, presentations, spreadsheets, textual content, mapping and geographic information, address information, profile information, and a variety of other electronic data, or any combination thereof.

Database 106 may be a separate component or an integrated component. For example, database 106 may be separate from the user device 102 and/or remote device 104. Additionally or alternatively, database 106 may be integrated into the user device 102, such that the user's healthcare data, parameters associated with the user, predictive models, etc. are stored in the user device 102. Database 106 may be included in the system 100. Alternatively, database 106 may be located remotely from the system 100. Database 106 may include computing components (e.g., database management system, database server, etc.) configured to receive and process requests for data stored in memory devices of database 106 and to provide data from database 106.

System 100 may also include network 108, which may facilitate communications between a user device 102, a remote device 104, database 106, and/or server 110. In some exemplary embodiments, network 108 may include any combination of communications networks. For example, network 108 may include the Internet and/or any type of wide area network, an intranet, a metropolitan area network, a local area network (LAN), a wireless network, a cellular communications network, a Bluetooth network, or any other type of electronics communications network, etc.

System 100 may also include a server 110. Server 110 may be an external servicer, a web server, a cloud storage server, a social network service (SNS) server, or an application programming interface (API) server. Server 110 can enable communications between a user device 102, a remote device 104, database 106, and/or network 108.

The components and arrangement of the components included in system 100 may vary. Thus, system 100 may further include other components that perform or assist in the performance of one or more processes consistent with the disclosed embodiments. Further, system 100 may include any number of user devices 102, remote devices 104, and/or databases 106. Although exemplary functions may be described as performed by a particular component of system 100 for ease of discussion, some or all disclosed functions of that particular component may interchangeably be performed by one or more of user device 102, remote device 104, database 106, network 108, and/or server 110.

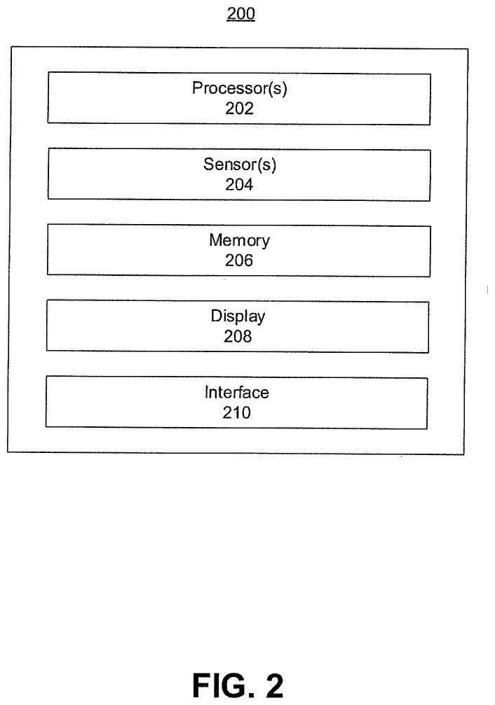

FIG. 2 illustrates an exemplary device 200 for providing a personalized blood test. By way of example, device 200 may be a user device 102 or a remote device 104 of FIG. 1. The arrangement and number of components in device 200 are provided for purposes of illustration. Additional arrangements, number of components, and other modifications may be made, consistent with the present disclosure.

Device 200 may include one or more processors 202 for executing instructions. Device 200 may also include one or more sensor(s) 204. Sensor(s) 204 may include one or more image sensors, or any other types of sensors configured to capture an image and/or a video of a user. For example, sensor(s) may include one or more cameras.

As further illustrated in FIG. 2, system 200 may include memory 206 configured to store data or one or more instructions and/or software programs that perform functions or operations when executed by the one or more processors 202. By way of example, memory 206 may include Random Access Memory (RAM) devices, NOR or NAND flash memory devices, Read Only Memory (ROM) devices, etc.

System 200 may also include one or more displays 208 for displaying data and information. Display 208 may be implemented using devices or technology, such as a cathode ray tube (CRT) display, a liquid crystal display (LCD), a plasma display, a light emitting diode (LED) display, a touch screen type display, a projection system, and/or any other type of display known in the art.

System 200 may also include at least one interface 210. Interface 210 may allow software and/or data to be transferred between device 200, user device 102, remote device 104, database 106, network 108, server 110, and/or other components. Examples of interface 210 may include a modem, a network interface (e.g., an Ethernet card or a wireless network card), a communications port, a PCMCIA slot and card, a cellular network card, etc. Interface 210 may transfer software and/or data in the form of signals, which may be electronic, electromagnetic, optical, or other signals capable of being transmitted and received by interface 210. Interface 210 may transmit or receive these signals using wire, cable, fiber optics, radio frequency (“RF”) link, Bluetooth link, and/or other communications channels.

FIG. 3A illustrates an exemplary graphical user interface (GUI) 310 displayed on an exemplary device 300 in accordance with the embodiments of the present disclosure. Device 300 may be the user device 102 and/or the remote device 104. As discussed above, device 300 may be configured to execute an application 312. Accordingly, GUI 310 may display the application 312 running on the device 300. For example, GUI 310 may display one or more healthcare data and/or one or more physiological parameters associated with the user of the user device 102. One or more healthcare data and/or one or more user inputs associated with the user may include an image of the user and/or omics data associated with the user, including at least one of clinome, phenome, exposome, genome, proteome, microbiome, pharmacome, or physiome. Device 300 may further include one or more sensors 314. Sensor(s) 314 may include one or more image sensors, or any other types of sensors configured to capture an image and/or a video of a user. For example, sensor(s) 314 may include one or more cameras.

Clinome may refer to any clinical information associated with the user. For example, clinome may include the user's physiological conditions or medical procedures undertaken. Phenome may refer to any physiological information or observable characteristics associated with the user. For example, phenome may include physical appearance or properties/traits of the user or behavior associated with the user. Exposome may refer to the environmental characteristics, to which the user is exposed. For example, exposome may include environmental factors such as climate factors, social capital, and/or exposure to stress, contaminants, radiation, infections, viruses. Exposome may further include lifestyle factors, diet, and/or physical activity. Exposome associated with the user may be determined based on the location of the user. Genome may refer to the genetic makeup of the user. For example, genome may include information associated with the set of nucleotides that make up all of the chromosomes of the user. Genome may not only include genetic information of the user, but also include genetic information of the user's family members or relatives. Proteome may refer to the set of proteins expressed by the user's genome. Proteome of the user may be determined based on testing performed by the laboratory, including blood tests. Microbiome may refer to the internal ecosystem of bacteria located within the body of the user. Microbiome associated with the user may also be determined based on testing performed by the laboratory, including blood tests. Pharmacome may refer to a list of prescriptions, medications, and/or supplements taken by the user. Finally, physiome may refer to the user's physiological state or behavior. For example, physiome may include information associated with the user's activity levels or vitals, such as number of steps taken each day, sleeping patterns, heart rate measurements, blood pressure measurements, etc.

While FIG. 3A only shows a list of clinome, phenome, exposome, genome, proteome, microbiome, pharmacome, and physiome associated with the user, healthcare data, user input, and/or parameters displayed on GUI 310 are not limited to these omics data. For example, healthcare data and/or parameters displayed may further include various physiological information, including skin tone, skin color, presence of wrinkles, scars, acne, or bags, receding hairlines, dental hygiene, or facial symmetry. Other parameters displayed may further include the user's gender, age, ethnicity, race, weight, height, or body mass index (BMI). By way of example, one or more processors 202 may request the user to take an image of the user, via the sensor(s) 314. One or more processors 202 may be able to predict one or more physiological parameters associated with the user based on the image. In addition, one or more processors 202 may be able to calculate optimal ranges associated with one or more health data associated with the user. Optimal ranges may be calculated, via a machine-learning algorithm, based on the parameters associated with the user. As discussed in detail below, one or more processors 202 may be able to implement a trained neural network model in order to predict parameters associated with the user. Additionally or alternatively, one or more processors 202 may be able to access the database 106 in order to obtain healthcare data and/or physiological parameters associated with the user.

User may be able to select one or more of the omics data displayed on GUI 310 and manually input additional information associated with the user. Additionally or alternatively, device 300 may be able to automatically receive data associated with the user from clinicians, doctors, hospitals, pharmacies, wearable devices, and/or other remote devices in electronic communication with the device 300.

As illustrated in FIG. 3B, GUI 310 may further display one or more predictions associated with the user. Predictions may include one or more future health-related information associated with the user. For example, predictions may include at least one of a number of future healthcare visits the user will have, risks for mortality causes, microbial diversity, healthiest location to live, a number of steps the user will take per day, future potential for weight gain, risk of allergies, or future sleep patterns.

By way of example, one or more healthcare data and parameters associated with the user may be transmitted to a remote device 104. The healthcare data associated with the user may be filtered by the parameters associated with the user to obtain personalized health data. For example, the user's blood test results may be filtered by the user's gender, age, and ethnicity in order to optimize the blood test results. The optimized blood test results may be customized to the user's physiological parameters, thereby providing personalized blood test results. Based on the user's personalized health data, one or more predictive models may be provided that is configured to predict future health-related information. The one or more predictive models may be transmitted back to the device 300 and stored in database 106 and/or memory 206. Based on the predictive models, one or more processors 202 may be able to predict future health-related information associated with the user and display the predictions on GUI 310 executing the application 312. User may be able to select from the list of available predictions on GUI 310.

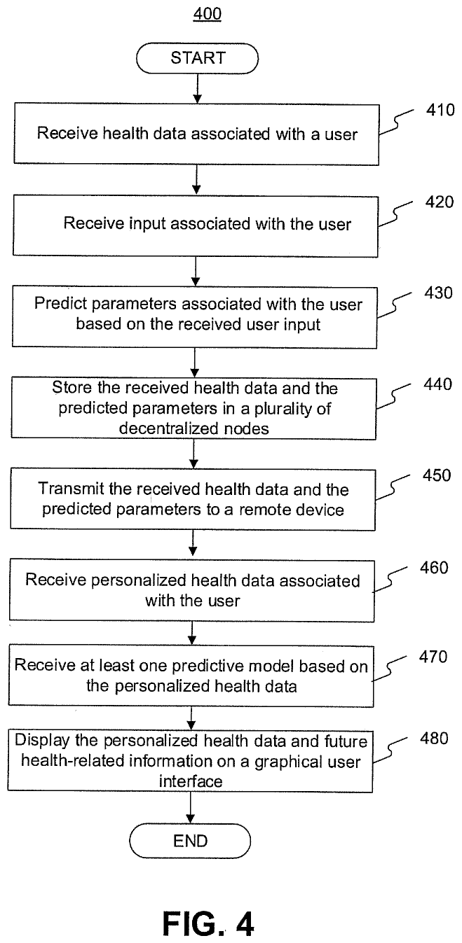

Reference is now made to FIG. 4, which is a flowchart of an exemplary process of providing a personalized blood test. Process 400 may be implemented, for example, on user device 102 with or without communications with database 106 via network 108 and/or server 110. The order and arrangement of steps in process 400 is provided for purposes of illustration. As will be appreciated from this disclosure, modifications may be made to process 400 by, for example, adding, combining, removing, and/or rearranging one or more steps of process 400. It is contemplated that in performing process 400, notifications, information, message, images, graphical user interface, etc. may be displayed, for example, on user device 102 and/or remote device 104. Further, it is contemplated that in performing process 400, users may make one or more selections from a GUI displayed on display 208. In addition, it is contemplated that in performing process 400, information or data may be accessed, retrieved, or stored in one or more of memory 206 or database 106. One or more of memory 206 or database 106, by way of example, may be associated with one or more of sensor(s) 204, user device 102, and/or remote device 104.

As shown in FIG. 4, process 400 may include step 410 of receiving health data associated with the user. In some embodiments, the health data may include test results from hospitals or laboratories. For example, health data associated with the user may be blood test results of the user. In some embodiments, the user may be prompted on the user device 102 to provide health data associated with the user. In other embodiments, health data associated with the user may be, upon request or automatically, transmitted from hospitals, clinicians, technicians, doctors, pharmacists, user wearable devices, or any combination thereof associated with the user.

Process 400 may further include step 420 of receiving an input associated with the user. The input associated with the user can be received from user device 102 associated with the user. In some embodiments, for example, the input associated with the user may be an image of the user. For example, device 102 can have its own photo taking function, can store images received from other devices, and/or can access images in other devices. Such accessible images may be taken by another device. In some embodiments, the user may be prompted on the user device 102 to capture an image of the user. Accordingly, the user may capture one or more images of the user real-time and upon request. Additionally or alternatively, the user may be able to select one or more of the images stored in the device 102. The user may also be able to select images stored in other devices. The image can be a digital image of the user, including at least a part of the user's facial image. Additionally or alternatively, the image could be a fully body image, upper body image, or facial image. Other suitable types of images can be understood by one of skill in the art. In other embodiments, the input associated with the user can include omics data. As discussed above, omics data, for example, may include clinome, phenome, exposome, genome, proteome, microbiome, pharmacome, and/or physiome.

Upon receiving the user input, process 400 may continue to step 430 of predicting parameters associated with the user based on the user input. In some embodiments, the user input may be sent, via server 110, to a physiological parameter determination block (not shown). The physiological parameter determination block may be a component within the user device 102. In other embodiments, the physiological parameter determination block may be a component separate from the user device 102. If the user input includes an image of the user, for example, the physiological parameter determination block may include an image processor and a predictor. As discussed in further detail below, the image processor can be configured to analyze the image of the user and predict one or more parameters associated with the user, including gender, age, ethnicity, weight, height, BMI, or any other physiological parameters. The image processor and/or the predictor may implement machine learning algorithms to predict parameters associated with the user, such as a trained neural network model or a deep learning convolutional neural network model.

Process 400 may further include step 440 of storing health data and/or parameters associated with the user in a plurality of decentralized nodes. For example, health data and/or parameters associated with the user may be stored in each user's device 102 such that other devices, such as the remote device 104, may not have immediate access to the user's stored data. Step 440 of storing health data and/or parameters associated with the user in a plurality of decentralized nodes may be performed using block chain technology. Data, including health data and/or parameters, associated with the user may be encrypted before being stored in the plurality of decentralized nodes.

Furthermore, process 400 may include step 450 of transmitting health data and/or parameters associated with the user to a remote device 104. Health data and/or parameters associated with the user may be transmitted to the remote device 104 via network 108 and/or server 110. In some embodiments, the user may be prompted on the user device 102 to allow transmission of health data and/or parameters associated with the user to the remote device 104. For example, the user may request to join a clinical trial, and thus, be prompted to transmit health data and/or parameters associated with the user to the remote device 104 responsible for aggregating each participants' data. In some embodiments, health data and/or parameters associated with the user that is transmitted to the remote device 104 may be aggregated and used to train a predictive model, such as a neural network model or a deep learning convolutional neural network model.

Process 400 may further include step 460 of receiving personalized health data. In some embodiments, personalized health data may comprise user's health data that has been filtered by one or more parameters associated with the user. Parameters may include, among other things, skin tone, skin color, presence of wrinkles, scars, acne, or bags, receding hairlines, dental hygiene, or facial symmetry. Other parameters displayed may further include the user's gender, age, ethnicity, race, weight, height, or body mass index (BMI). In some aspects, personalized health data may comprise personalized blood test results. User's blood test results may be filtered by one or more parameters associated with the user, such as the user's age, gender, and ethnicity, thereby providing personalized blood test results. This may improve the blood test results' accuracy and precision in determining a physiological condition of the user.

Process 400 may continue to step 470 of receiving at least one predictive model based on the personalized health data. In some embodiments, the at least one predictive model may be configured to predict future health-related information of the user. By way of example, future health-related information of the user may include, among other things, at least one of a number of future healthcare visits the user will have, risks for mortality causes, microbial diversity, healthiest location to live, a number of steps the user will take per day, future potential for weight gain, risk of allergies, or future sleep patterns. The predictive models may include trained neural network models or deep learning convolutional neural network (DNN) models configured to predict future health-related information of the user.

Moreover, process 400 may include step 480 of displaying personalized health data and/or future health-related information on GUI of a device. By way of example, the user's personalized health data and/or future health-related information may be displayed on GUI 310 of device 300. Device 300 may be the user device 102. In some embodiments, the user may be able to interact with or manipulate the information displayed on GUI 310 of device 300. By way of example, the user may be able to share or send the displayed information to other remote devices.

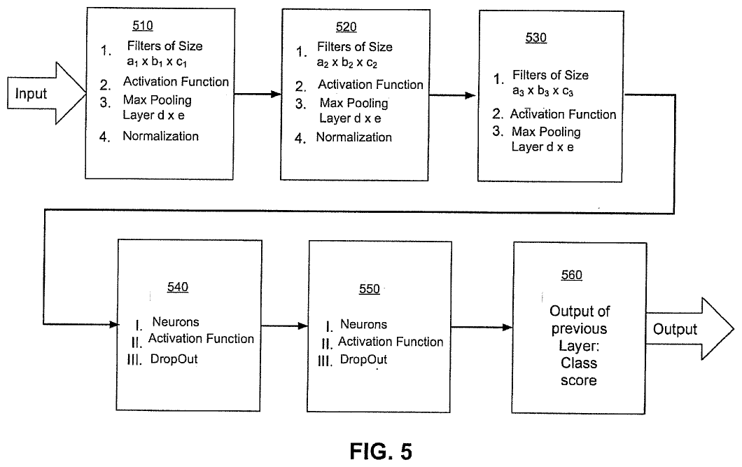

Reference is now made to FIG. 5, which shows an exemplary algorithm for processing an image of the user to predict an age group classification associated with the user. A number of layers of the algorithm can vary. Age group classification is a factor for predicting physiological parameters like BMI value accurately and reliably. A deep-learning based approach can be an effective machine-learning method to handle unconstrained imaging conditions most likely encountered in selfie images. In some embodiments, a DNN algorithm may be adopted to handle unconstrained images.

Layer 510 can be configured to be a convolutional layer. In this layer, input image may be convoluted with filters. In some embodiments, the input image may be in three color channels (e.g., Red, Green, Blue). Each of the filters can be configured to be a matrix pattern of a size aᵢ×bᵢ×cᵢ. For example, each of the filters can be configured to be a matrix pattern in the size of 3×7×7. Thereafter, activation function, such as a Rectified Linear Unit (ReLU), can be applied to every pixel of the image in various color channels. As a result of ReLU, an image pixel matrix can be derived. The image pixel matrix can further be downsized in the step of Max Pooling by a pre-defined filter size d×e. For example, the filter can be configured to be a square, e.g., 3×3. Other downsizing layers may include AvgPool, etc. The downsized data can then be converted to a two-dimensional data and be normalized, for example by Batch normalization. As a result of normalization, the matrix becomes a well-behaved matrix with mean value approximately equal to 0 and variance approximately equal to 1. As other convolutional layers, layer 520 and layer 530 can be configured to apply similar functions into the image pixel matrix.

In layer 540, the convoluted image pixel matrix may be applied to a fully connected layer for liner transformation. The image pixel matrix may be multiplied by a predetermined number of neurons so that the image pixel matrix is converted into a reduced dimensional representation with a predetermined number of values. In DropOut step, the reduced dimensional representation is defined by probability value. Layer 550 can be configured to apply similar functions into the reduced dimensional representation.

The layer 560 can be another fully connected layer. In layer 560, the matrix can be reduced to, for example, four final outputs, e.g., height, weight, age group classification, and gender. The outputs may be the predictions of the neural network algorithm, which can be compared with values of the parameters associated with images for further training purpose of the algorithm.

In some embodiments, age estimation and prediction may be based on calculation of ratios between measurements of parameters of various facial features. After facial features (e.g., eyes, nose, mouth, chin, etc.) are localized and their sizes and distances in between are measured, ratios between these facial feature measurement parameters may be determined and used to classify the user's face into an age group class according to empirical rules defined by physiological research.

In some embodiments, local features of a face can be used to represent facial images and Gaussian Mixture Model may be used to represent the distribution of facial patches. Robust descriptors can be used to replace pixel patches. In other embodiments, Gaussian Mixture Model can be replaced by Hidden-Markov Model, and super-vectors may be used to represent face patch distributions. In yet further embodiments, robust image descriptors may be used to replace local imaging intensity patches. Gabor image descriptor may be used along with a Fuzzy-LDA classifier, which may consider the possibility of one facial image belonging to more than one age group. In some embodiments, a combination of Biologically-Inspired Features and various manifold-learning methods may be used for age estimation. In some embodiments, Gabor and local binary patterns (LBP) may be used along with a hierarchical age classifier composed of Support Vector Machines (SVM) to classify the input image to an age-class followed by a support vector regression to estimate a precise age. Improved versions of relevant component analysis and locally preserving projections may be adopted. Those methods may be used for distance learning and dimensionality reduction with Active Appearance Models as an image feature as well. In some embodiments, LBP descriptor variations and a dropout Support Vector Machines (SVM) classifier can be adopted.

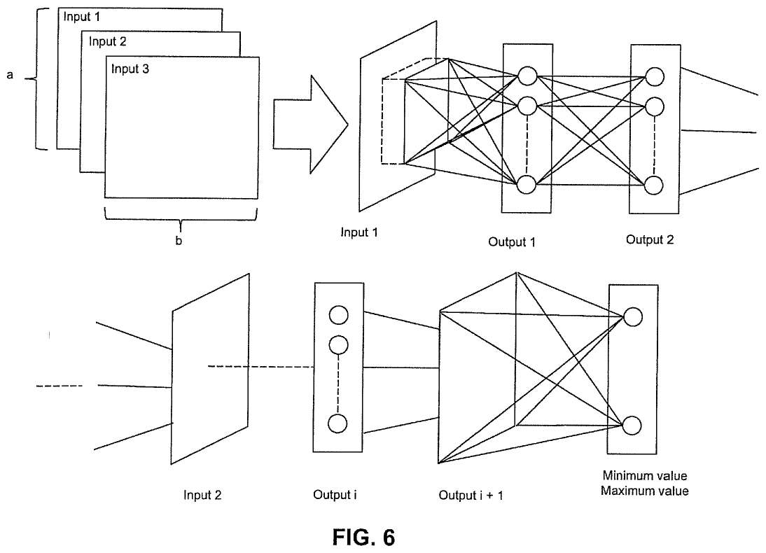

Reference is now made to FIG. 6, which shows an exemplary schematic diagram illustrating an exemplary regression DNN model to predict parameters, such as personalized blood test normal ranges to various analytes, of the user, in accordance with the embodiments of the present disclosure.

In some embodiments, for example, the model may include three parameter inputs, which can be, for example, any of clinome, phenome, exposome, genome, proteome, microbiome, pharmacome, or physiome, seventeen hidden layers, and/or two outputs of the three parameters inputs, such as personalized normal range of a first analyte and personalized normal range of a second analyte. Pre-trained transfer learning models can be used. Input data can be adjusted to have a resolution of a×b, e.g., 224×224. The first input layer can be a convolutional layer with predetermined size, for example a size of 96×7×7. The first hidden layer can be configured to be followed by a ReLU Activation, a Max Pooling Layer with an exemplary size of 3×3, a stride with an exemplary size of 2×2, and a batch normalization. The second input layer can be a convolutional layer with an exemplary size of 256×5×5. The second input layer can be configured to be followed by a ReLU Activation, a Max Pooling Layer with an exemplary size of 3×3, and a batch normalization. The third input layer can be a convolutional layer with an exemplary size of 384×3×3. The third input layer can be configured to be followed by a ReLU Activation and a Max Pooling Layer with an exemplary size 3×3. Other input layers can be configured in a similar way and therefore are not repeated here.

Within the seventeen input layers, the three input layers can be configured to be output layers, for example, fully connected layers. Output 6 (not shown in FIG. 6) can be configured to be the first output layer (Output 1) with neurons fully connected to the previous layer, followed by a ReLU Activation and a DropOut function. Output 7 (not shown in FIG. 6) can be configured to be the second output layer (Output 2) with neurons fully connected to the previous layer, followed by a ReLU Activation and DropOut layer. Output layer 8 (not shown in FIG. 6) can be configured to be the third output layer (Output 3) with neurons fully connected to output layer 7, yielding the un-normalized class values.

The regression DNN algorithm disclosed in FIG. 6 can be applied to build separate models on personalized blood test normal ranges of multiple analytes. Personalized blood test normal ranges can be returned to the user device 102, and results can be presented or displayed to the user associated with the device 102, for example on GUI 310 of device 300.

In some embodiments, the DNN may be a supervised neural network. Input data, such as input health data, may be configured to be bound with label information or meta data representing the content of the data. In a personalized blood analyte normal range prediction application, such meta data can be a suggested blood test normal range of multiple analytes of the person associated with the data. For each data used in the training process, values of the person of the data may be associated. Therefore, the DNN may receive feedback by comparing predicted blood test normal range values to associated suggested values of blood test normal range to further improve its prediction algorithm. To serve the supervised training purpose in accordance with aspects of the disclosure, input health data associated with suggested blood test normal range of multiple analytes in the training database may need to be at a large scale. For example, daily exposome data may comprise more that several years of history.

In some embodiments, output layers, such as fully connected layers, can express a set of features describing the input data. Accordingly, the feature vectors in the output layers may comprise more data in them than the original raw pixel values of the input data. Many processes can be done on these feature vectors. In some embodiments, a NiN can be used as a Conventional Neural Network known to work well on image processing. Many other neural networks can be understood and chosen by a skill in the art without violating the principle stated in the embodiments of the disclosure.

In some embodiments, Stochastic Gradient Descent (SGD) may be applied to train the NiN. This learning algorithm may have two learning algorithms set by the user: Learning Rate and Momentum. These parameters are usually hand-tuned in the beginning iterations of SGD to ensure the network is stable. Training the regression NiN model can start from the parameters pre-set.

In some aspects, the learning rates may not be adjusted over the duration of the batches. The mechanism of learning can be used to optimize the error between labeled blood test normal range of analyte values associated with the input data and the outputs, estimated blood test normal range of analyte values of the user associated with the data, of the neural network. In mathematical optimization problem, this mechanism of learning may be a loss function, which can also be cost function or objective function. A typical loss function for regression may be Mean Absolute Error (MAE) given by equation as below.

$$MAE ( x , y ) = \frac{1}{n} {\sum }_{i = 1}^{n} \vert {x}_{i} - {y}_{i} \vert$$

where x is the observed output of the neural network, and y is label information associated with the facial image (i.e., weight and height value of the subject person), and n is the number of images in the batch or dataset. MAE is not influenced by positive or negative errors, namely the direction of the error. This means the model can either over or under estimate weight and height. In some embodiments, this loss function model can also be Root Mean Squared or Mean Squared Error.

In some embodiments, the error level of the trained algorithm may decrease as the amount of data fed into the algorithm increases. After a certain amount is data are processed to train the algorithm, the error level may reduce dramatically. Once the error level of the training algorithm has reduced dramatically, the error level may be limited to a range of tolerance indicating that the trained algorithm is satisfactory for physiological parameters predictions.

Other embodiments of the disclosure will be apparent to those skilled in the art from consideration of the specification and practice of the embodiments disclosed herein. It is intended that the specification and examples be considered as exemplary only, with a true scope and spirit of the invention being indicated by the following claims.

1. A computer-implemented method of providing a personalized blood test, the method comprising:
receiving, from a digital device, health data associated with a user, the health data comprising blood test results;
receiving, from the digital device, an input associated with the user;
predicting, via a trained neural network model, parameters associated with the user based on the received user input;
storing, using block chain, the received health data and the predicted parameters in a plurality of decentralized nodes;
transmitting the received health data and the predicted parameters to a remote device;
receiving, from the remote device, personalized health data associated with the user, wherein the personalized health data comprises the received health data filtered by the predicted parameters;
receiving, from the remote device, at least one predictive model based on the personalized health data, wherein the predictive model is configured to predict future health-related information; and
displaying the personalized health data and the future health-related information on a graphical user interface of the digital device.

2. The computer-implemented method of claim 1, wherein the predicted parameters associated with the user comprise at least one of gender, age, ethnicity, weight, height, or body mass index.

3. The computer-implemented method of claim 1, wherein the user input comprises at least one of an image of the user, clinome, phenome, exposome, genome, proteome, microbiome, pharmacome, or physiome.

4. The computer-implemented method of claim 1, wherein the personalized health data comprises personalized blood test results that are filtered by gender, age, and ethnicity associated with the user.

5. The computer-implemented method of claim 1, wherein the future health-related information comprises at least one of a number of future healthcare visits the user will have, risks for mortality causes, microbial diversity, healthiest location to live, a number of steps the user will take per day, future potential for weight gain, risk of allergies, or future sleep patterns.

6. The computer-implemented method of claim 1, further comprising displaying, on the graphical user interface, optimal range associated with the personalized health data, wherein the optimal range is calculated, via a machine-learning algorithm, based on the predicted parameters associated with the user.

7. The computer-implemented method of claim 1, further comprising:
aggregating the received health data associated with the user with shared health data received from other users; and
updating the predictive model based on the aggregation.

8. The computer-implemented method of claim 1, further comprising:
generating a reward to the user in response to determining that the received health data and the predicted parameters are transmitted to the remote device; and
displaying the reward on the graphical user interface.

9. The computer-implemented method of claim 1, further comprising encrypting the received health data and the predicted parameters before storing the received health data and the predicted parameters in the plurality of decentralized nodes.

10. The computer-implemented method of claim 1, wherein the digital device comprises at least one of a computer, a laptop, a smartphone, a tablet, or a smartwatch.

11. A non-transitory computer-readable medium comprising a set of instructions that are executable by at least one processor of a device to cause the device to perform operations, comprising:
receiving, from a digital device, health data associated with a user, the health data comprising blood test results;
receiving, from the digital device, an input associated with the user;
predicting, via a trained neural network model, parameters associated with the user based on the received user input;
storing, using block chain, the received health data and the predicted parameters in a plurality of decentralized nodes;
transmitting the received health data and the predicted parameters to a remote device;
receiving, from the remote device, personalized health data associated with the user, wherein the personalized health data comprises the received health data filtered by the predicted parameters;
receiving, from the remote device, at least one predictive model based on the personalized health data, wherein the predictive model is configured to predict future health-related information; and
displaying the personalized health data and the future health-related information on a graphical user interface of the digital device.

12. The non-transitory computer-readable medium of claim 11, wherein the predicted parameters associated with the user comprise at least one of gender, age, ethnicity, weight, height, or body mass index.

13. The non-transitory computer-readable medium of claim 11, wherein the user input comprises at least one of an image of the user, clinome, phenome, exposome, genome, proteome, microbiome, pharmacome, or physiome.

14. The non-transitory computer-readable medium of claim 11, wherein the personalized health data comprises personalized blood test results that are filtered by gender, age, and ethnicity associated with the user.

15. The non-transitory computer-readable medium of claim 11, wherein the future health-related information comprises at least one of a number of future healthcare visits the user will have, risks for mortality causes, microbial diversity, healthiest location to live, a number of steps the user will take per day, future potential for weight gain, risk of allergies, or future sleep patterns.

16. The non-transitory computer-readable medium of claim 11, wherein the set of instructions further cause the device to display, on the graphical user interface, optimal range associated with the personalized health data, and wherein the optimal range is calculated, via a machine-learning algorithm, based on the predicted parameters associated with the user.

17. The non-transitory computer-readable medium of claim 11, wherein the set of instructions further cause the device to:
aggregate the received health data associated with the user with shared health data received from other users; and
update the predictive model based on the aggregation.

18. The non-transitory computer-readable medium of claim 11, wherein the set of instructions further cause the device to:
generate a reward to the user in response to determining that the received health data and the predicted parameters are transmitted to the remote device; and
display the reward on the graphical user interface.

19. The non-transitory computer-readable medium of claim 11, wherein the set of instructions further cause the device to encrypt the received health data and the predicted parameters before storing the received health data and the predicted parameters in the plurality of decentralized nodes.

20. The non-transitory computer-readable medium of claim 11, wherein the digital device comprises at least one of a computer, a laptop, a smartphone, a tablet, or a smartwatch.
